(19) United States
(12) Patent Application Publication
(10) Pub. No.: US 2025/0344974 A1
(43) Pub. Date: Nov. 13, 2025

(54) Blood Draw Device With Kink Prevention Catheter Support

(71) Applicant: Becton, Dickinson and Company, Franklin Lakes, NJ (US)

(72) Inventors: Jonathan Karl Burkholz, Salt Lake City, UT (US); Sridhaar Nandakumar, Chennai (IN); Prasad Govindaraj, Coimbatore (IN); Meenakshi Sankar G, Tirunelveli (IN); Mukilan Balamurugan, Theni (IN)

(21) Appl. No.: 19/199,607

(22) Filed: May 6, 2025

Related U.S. Application Data
(60) Provisional application No. 63/644,545, filed on May 9, 2024.

(51) Int. Cl. A61B 5/15; A61M 25/00

(52) U.S. Cl. CPC A61B 5/150992; A61M 25/0043; A61M 2025/0063

(57) ABSTRACT

A blood draw device for advancing a catheter into an indwelling catheter includes an introducer having a housing, an advancement member movable relative to the housing to move the catheter between a first position where the catheter is disposed within the introducer, and a second position where a distal end portion of the catheter is disposed beyond the distal end portion of the introducer. One or more catheter support inserts are positioned between the advancement member and the distal end portion of the housing, with each of the catheter support inserts defining a passageway therein that receives the catheter to provide support thereto. Each catheter support insert includes first and second end discs having an opening through which the catheter is routed and a flexible hinge member disposed between the first and second end discs and flexes so as to vary a distance between the first and second end discs.

### CROSS-REFERENCE TO RELATED APPLICATION

The present application claims priority to U.S. Provisional Patent Application No. 63/644,545 entitled “Blood Draw Device with Kink Prevention Catheter Support” filed May 9, 2024, the entire disclosure of which is hereby incorporated by reference.

### BACKGROUND OF THE INVENTION

### Field of the Invention

The present invention is directed to a blood draw device for introducing a catheter into an indwelling catheter of a vascular access device.

### Background of the Invention

Catheters are frequently utilized to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples.

Recent developments in the PIVC field have led to the emergence of technologies designed to facilitate blood draw using an in-dwelling PIVC and accompanying blood draw device. The main method by which these devices work is by employing an introducer for inserting a catheter, probe, tube, or other instrument through the catheter lumen of the PIVC, with the introducer being attached to a catheter adapter of the PIVC that provide for insertion of the catheter. For example, the catheter adapter may include a needleless access connector thereon by which the catheter may be introduced to provide access to the PIVC and into the patient's vasculature. A syringe and/or vacutainer may then be used collect blood samples without needing to subject the patient to additional needle sticks.

The introducer of a blood draw device typically includes a housing, a catheter movable within the housing so as to be extendable out therefrom for advancement into the in-dwelling PIVC, and an advancement member or advancement member that may be actuated by an operator relative to the housing. That is, the advancement member may be moved distally by the operator to cause a corresponding movement of the catheter relative to the housing, such that the catheter may be advanced out from the distal end of the housing and into the in-dwelling PIVC. However, during deployment of the catheter of the blood draw device, especially when the distal end portion of the catheter reaches a curved region of the PIVC (near where the PIVC enters the skin) and is caused to curve back and forth as it passes along the vein, the catheter of the blood draw device is subject to a column load which can cause bending, kinking, and/or deformation of the catheter. As the catheter bends, it can move in random directions creating a sinusoidal wave, which then flattens against the sidewall of the housing of the blood draw device as the force increases. Additional force applied to the catheter by further movement of the advancement member can then cause the catheter to double back on itself and collapse—i.e., to “buckle.”

Accordingly, a need exists for a blood draw device and introducer thereof that provides for advancement of a catheter into the PIVC while minimizing the potential for bending, kinking, or deformation of the catheter. It is desirable for such kink prevention features to be easily incorporated into the blood draw device, such that the blood draw device may be manufactured at scale and at low cost.

### SUMMARY OF THE INVENTION

Provided herein is a blood draw device for advancing a catheter into an indwelling catheter of a vascular access device. The blood draw device includes an introducer comprising a housing having a proximal end portion and a distal end portion, the distal end portion coupleable to the vascular access device, and with the housing defining an inner volume configured to movably receive the catheter. The blood draw device also includes an advancement member movably coupled to the housing and configured to move relative to the housing to move the catheter between a first position, in which the catheter is disposed within the introducer, and a second position, in which a distal end portion of the catheter is disposed beyond the distal end portion of the introducer, such that at least a first portion of the catheter may be disposed within the indwelling catheter. The blood draw device further includes one or more catheter support inserts positioned within the inner volume and positioned between the advancement member and the distal end portion of the housing, each of the one or more catheter support inserts defining a passageway therein that receives the catheter to provide support to a length of the catheter that extends between the advancement member and the distal end portion of the housing. Each of the one or more catheter support inserts comprises a first end disc and a second end disc each including an opening therein through which the catheter is routed, and a hinge member disposed between the first end disc and the second end disc and connecting the first end disc to the second end disc, the hinge member being flexible so as to vary a distance between the first end disc and the second end disc.

In some embodiments, the hinge member comprises a living hinge formed integrally with the first end disc and the second end disc.

In some embodiments, the living hinge comprises a first leg including a first end and a second end and a second leg including a first end and a second end, wherein the first end of the first leg is joined to the first end disc and the first end of the second leg is joined to the second end disc and wherein the second end of the first leg and the second end of the second leg are joined together, with the first leg and the second leg forming a V-shape when in an at-rest position.

In some embodiments, each of the first leg and the second leg includes an opening formed therein through which the catheter is routed, and wherein the openings in the first and second legs and the openings in the first and second end discs collectively define the passageway of the catheter support insert.

In some embodiments, for a respective catheter support insert of the one or more catheter support inserts that is nearest the advancement member, the advancement member engages the catheter support insert during movement thereof when moving the catheter from the first position to the second position, with the advancement member engaging and moving the catheter support insert with respect to the housing.

In some embodiments, as the advancement member moves the catheter support insert with respect to the housing, the hinge member flexes to transition the catheter support insert from an extended state to a compressed state, thereby shortening a length of the catheter support insert.

In some embodiments, in the extended state, the catheter support insert has a length of 5 mm.

In some embodiments, the advancement member includes a first portion comprising an engagement portion movable along a top surface of the housing and a second portion positioned at least partially within the inner volume and joined to the first portion through a slot formed in the top surface of the housing, wherein the second portion engages the catheter support insert as the advancement member is moved distally along the housing.

In some embodiments, the advancement member comprises a telescopic cylinder configured to slide distally into the inner volume of the housing, the telescopic cylinder including a fitting at a distal end thereof that engages the catheter support insert as the telescopic cylinder is moved distally into the housing.

In some embodiments, the one or more catheter support inserts comprises a plurality of catheter support inserts spaced apart along a length of the catheter between the advancement member and the distal end portion of the housing.

In some embodiments, the plurality of catheter support inserts are spaced apart equidistantly along the length of the catheter.

In some embodiments, with the catheter in the second position, the advancement member moves the plurality of catheter support inserts to the distal end portion of the housing, with the plurality of catheter support inserts in contact with each other in a stacked arrangement.

In some embodiments, a length of the stacked arrangement of the plurality of catheter support inserts is between 11.0 and 12.0 mm.

In some embodiments, the housing comprises an inner surface including one or more bump projections formed thereon, with a respective bump projection of the one or more bump projections configured to engage a respective catheter support insert of the one or more catheter support inserts to retain the catheter support insert in-place within the housing.

In some embodiments, the catheter support insert is pushed past the bump projection upon the advancement member engaging and moving the catheter support insert, as the advancement member moves the catheter from the first position to the second position.

Another blood draw device for advancing a catheter into an indwelling catheter of a vascular access device is provided that includes an introducer comprising a housing having a proximal end portion and a distal end portion, the distal end portion coupleable to the vascular access device, and with the housing defining an inner volume configured to movably receive the catheter. The blood draw device also includes an advancement member movably coupled to the housing and configured to move relative to the housing to move the catheter between a first position, in which the catheter is disposed within the introducer, and a second position, in which a distal end portion of the catheter is disposed beyond the distal end portion of the introducer, such that at least a first portion of the catheter may be disposed within the indwelling catheter. The blood draw device further includes a plurality of catheter support inserts positioned within the inner volume between the advancement member and the distal end portion of the housing, the plurality of catheter support inserts spaced apart along a length of the catheter, with each of the plurality of catheter support inserts defining a passageway therethrough that receives the catheter. With the advancement member moved distally relative to the housing to move the catheter from the first position to the second position, the advancement member pushes the plurality of catheter support inserts to the distal end portion, with the plurality of catheter support inserts positioned in a stacked arrangement.

In some embodiments, each of the plurality of catheter support inserts comprises a compressible member that transitions from an extended state to a compressed state when a distally directed force is applied thereto.

In some embodiments, with the plurality of catheter support inserts positioned in the stacked arrangement, all of the plurality of catheter support inserts are in contact with each other and are in the compressed state.

In some embodiments, a length of the stacked arrangement of the plurality of catheter support inserts is between 11.0 and 12.0 mm.

In some embodiments, each of the plurality of catheter support inserts comprises a first end disc and a second end disc each including an opening therein through which the catheter is routed, and a living hinge disposed between the first end disc and the second end disc and connecting the first end disc to the second end disc, the living hinge being actuatable to transition the catheter support insert from the extended state to the compressed state. The living hinge comprises a first leg and a second leg connected together, with each of the first leg and the second leg including an opening formed therein through which the catheter is routed, with the openings in the first and second legs and the openings in the first and second end discs collectively defining the passageway of the catheter support insert.

### BRIEF DESCRIPTION OF THE DRAWINGS

FIG. 1 is a perspective view of a blood draw device, according to an embodiment of the disclosure;

FIG. 2 is a top view of the blood draw device illustrated in FIG. 1;

FIG. 3 is an exploded view of the blood draw device of FIG. 1;

FIG. 4 is a perspective view of a first member of a housing included in the blood draw device of FIG. 1;

FIG. 5 is a perspective view of a second member of the introducer housing included in the blood draw device of FIG. 1;

FIG. 6 is a rear perspective view of the introducer housing formed by coupling the first member illustrated in FIG. 4 to the second member illustrated in FIG. 5;

FIG. 7 is a front perspective view of the introducer housing illustrated in FIG. 6;

FIG. 8 is a cross-sectional view of the introducer taken along the line 8-8 in FIG. 6;

FIG. 9 is a rear perspective view of a lock included in the blood draw device of FIG. 1;

FIG. 10 is an exploded perspective view a catheter, a secondary catheter, and an advancement member included in the blood draw device of FIG. 1;

FIG. 11 is a perspective view of the advancement member illustrated in FIG. 10;

FIG. 12 is a cross-sectional view of the blood draw device taken along the line 12-12 in FIG. 2;

FIG. 13 a perspective view of a catheter support insert included in the blood draw device of FIG. 1;

FIG. 14 is a side view of the catheter support of FIG. 13 in an extended state;

FIG. 15 is a side view of the catheter support of FIG. 13 in a compressed state;

FIG. 16 is a side cross-sectional view of the blood draw device of FIG. 1 in a first configuration, showing positioning of the advancement member and catheter support inserts;

FIG. 17 is a side cross-sectional view of the blood draw device of FIG. 1 in an intermediate configuration, showing positioning of the advancement member and catheter support inserts;

FIG. 18 is a side cross-sectional view of the blood draw device of FIG. 1 in a second configuration, showing positioning of the advancement member and catheter support inserts and

FIG. 19 is a perspective view of a blood draw device, according to another embodiment of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

The terms “first”, “second”, and the like are not intended to refer to any particular order or chronology, but refer to different conditions, properties, or elements.

The present invention is directed to a blood draw device for phlebotomy through a peripheral intravenous line or catheter, with the blood draw device operable to advance a flexible tube or catheter into the peripheral intravenous line and subsequently draw blood or administer a drug to a patient.

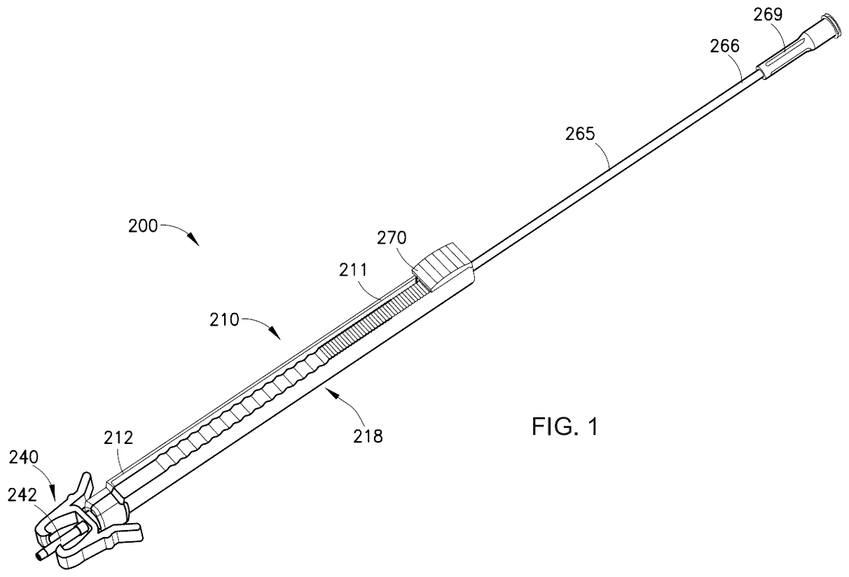

FIGS. 1-18 illustrate a blood transfer device 200 (i.e., blood draw device) and components thereof according to embodiments of the invention. The blood draw device 200 can be any suitable shape, size, or configuration and can be coupled to a PIVC (not shown in FIGS. 1-18), for example, via a lock and/or adapter. As described in further detail herein, a user can transition the blood draw device 200 from a first configuration to a second configuration to advance a catheter through an existing, placed, and/or indwelling PIVC (i.e., when the blood draw device 200 is coupled thereto) such that at least an end portion of the catheter is disposed in a distal position relative to the PIVC. Moreover, with peripheral intravenous lines each having a shape, size, and/or configuration that can vary based on, for example, a manufacturer of the PIVC and/or its intended usage, the blood draw device 200 can be arranged to allow the blood draw device 200 to be coupled to a PIVC having any suitable configuration and subsequently, to advance at least a portion of a catheter through the PIVC. In addition, the blood draw device 200 can be manipulated by a user to place a distal surface of the catheter a predetermined and/or desired distance beyond a distal surface of the PIVC to be disposed within a portion of a vein that receives a substantially unobstructed flow of blood.

As shown in FIGS. 1-3, the blood draw device 200 includes an introducer 210, a lock 240, a catheter 260, a secondary catheter 265, an advancement member 270, and one or more catheter support inserts 280. The introducer 210 can be any suitable shape, size, or configuration. For example, in some embodiments, the introducer 210 can be an elongate member having a substantially circular cross-sectional shape. In some embodiments, the shape of the introducer 210 and/or one or more features or surface finishes of at least an outer surface of the introducer 210 can be arranged to increase the ergonomics of the blood draw device 200, which in some instances, can allow a user to manipulate the blood draw device 200 with one hand (i.e., single-handed use).

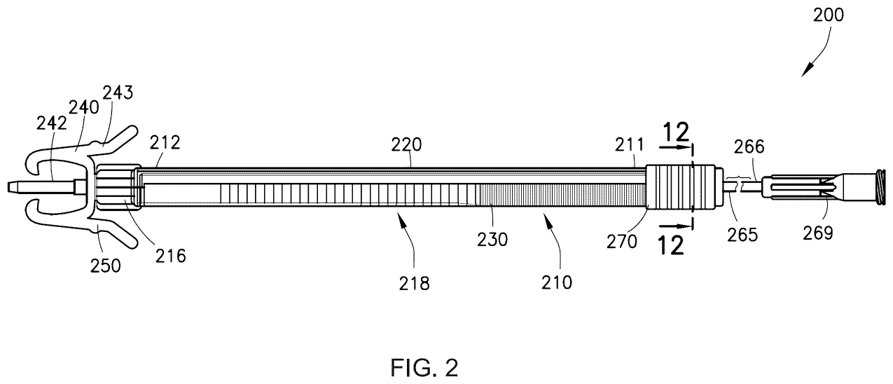

As shown in FIGS. 2-8, the introducer 210 of the blood draw device 200 includes a housing 218 having a first housing member 220 and a second housing member 230 that are coupled to collectively form the housing 218. The first housing member 220 includes a proximal end portion 221, a distal end portion 222, and an inner surface 223. The inner surface 223 has a first portion 224 and a second portion 225, with second portion 225 being a flange in one embodiment. The proximal end portion 221 of the first housing member 220, and more specifically, a proximal wall of the first housing member 220 defines a notch 226 configured to selectively receive a portion of the secondary catheter 265, as described in further detail herein.

The second housing member 230 has a proximal end portion 231, a distal end portion 232, an inner surface 233, and a top surface 235. As described above with reference to the first housing member 220, the proximal end portion 231 of the second housing member 230, and more specifically, a proximal wall of the second housing member 230 defines a notch 234 configured to selectively receive a portion of the secondary catheter 265.

The top surface 235 of the second housing member 230 includes a set of ribs 236 distributed along a length of the second housing member 230, with each rib 236 extending along a width of the second housing member 230. The ribs 236 formed by the top surface 235 of the second housing member 230 can be any suitable shape, size, and/or configuration. For example, the set of ribs 236 includes a first portion 237 having a first size and shape, and a second portion 238 having a second size and shape, different from the first size and shape. Each rib in the second portion 238 has a size and shape that is greater than the size and shape of each rib of the first portion 237.

As described in further detail herein, a portion of the advancement member 270 is configured to be advanced along the top surface 235 forming the set of ribs 236 as a user moves the advancement member 270 relative to the housing 218, which in turn, vibrates the advancement member 270 (and the catheter 260 coupled thereto). In some instances, this vibration can, for example, facilitate the advancing of the catheter 260 through a portion or the blood draw device 200, a portion of the PIVC, and/or a portion of the vasculature. In some instances, the greater size of the ribs of the second portion 238 can result in a larger amount of vibration as the advancement member 270 is moved along the top surface 235 (as described above). In some instances, the greater size of the ribs of the second portion 238 can result in an increase in a force otherwise sufficient to move the portion of the advancement member 270 along the top surface 235.

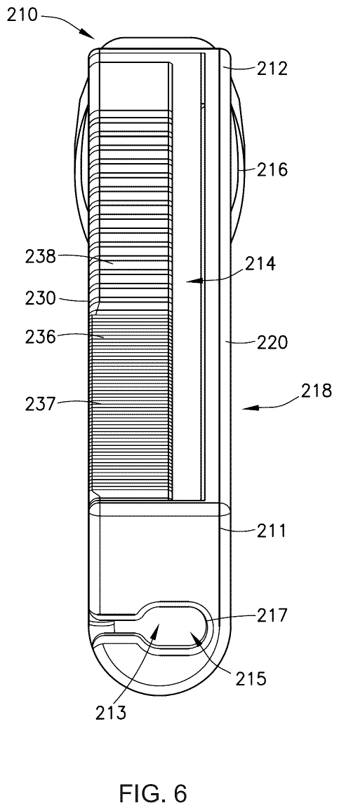

As shown in FIGS. 6-8, the first housing member 220 is configured to be coupled to the second housing member 230 to collectively form the housing 218. In some embodiments, forming the housing 218 from the first housing member 220 and the second housing member 230 can reduce undesirable variations in the shape and/or size of the inner surface 223 and 233 (e.g., due to draft angles and/or manufacturing tolerances) during manufacturing, which in some instances, can reduce a likelihood of kinks, bends, and/or deformations of the catheter 260 during use of the blood draw device 200. In some embodiments, forming the housing 218 from the first housing member 220 and the second housing member 230 can allow at least the inner surface 223 of the first housing member 220 to form a tortuous shape that would otherwise present challenges when manufacturing the housing 218 from a single work piece.

The first housing member 220 and the second housing member 230 collectively form a proximal end portion 211 and a distal end portion 212 of the housing 218 and collectively define an inner volume 213 of the housing 218. As shown in FIG. 6, the proximal end portion 211 of the housing 218 defines an opening 217. Specifically, the opening 217 is collectively formed and/or defined by the notch 226 of the first housing member 220 and the notch 234 of the second housing member 230. The arrangement of the proximal end portion 211 is such that a portion of the opening 217 defined by the notch 226 of the first housing member 220 has a first size and/or shape and a portion of the opening 217 defined by the notch 234 of the second housing member 230 has a second size and/or shape that is less than the first size and/or shape. In other words, a portion of the opening 217 is constricted, pinched, obstructed, and/or otherwise reduced. As described in further detail herein, the opening 217 is configured to receive a portion of the secondary catheter 265, which can be moved within the opening 217 from the larger portion of the opening 217 to the reduced portion of the opening 217 (e.g., the portion formed by the notch 234 of the second housing member 230) to obstruct, pinch, and/or clamp the secondary catheter 265.

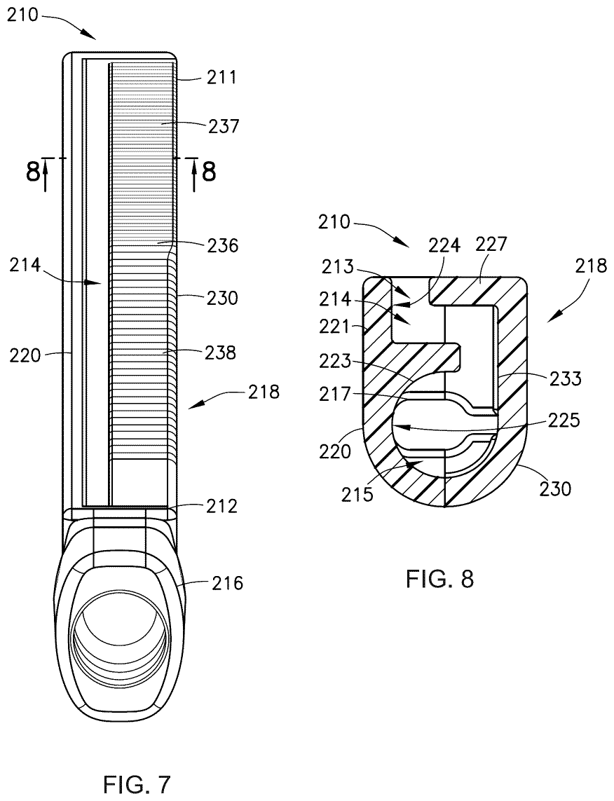

As shown in FIG. 7, the distal end portion 212 of the housing 218 includes and/or otherwise forms a coupler 216. In other words, the distal end portion 222 of the first housing member 220 and the distal end portion 232 of the second housing member 230 collectively form the coupler 216 at the distal end portion 212 of the housing 218. The coupler 216 can be any suitable shape, size, and/or configuration. For example, in this embodiment, the coupler 216 forms a set of threads, which can form a threaded coupling with an associated threaded portion of the lock 240, as described in further detail herein. Although not shown in FIG. 7, the distal end portion 211 of the housing 218 can include and/or can be configured to receive a seal (not shown) that can selectively seal and/or fluidically isolate the inner volume 213 of the housing 218 (at least from an open portion of the coupler 216). In use, the seal can be transitioned from a sealed or closed configuration to an open configuration to allow, for example, a portion of the catheter 260 to pass therethrough. In some embodiments, the seal can contact an outer surface of the catheter 260 to define a seal therebetween that is operable to limit and/or substantially prevent a back flow of fluid between the outer surface of the cannula and the seal.

The inner surface 223 of the first housing member 220 and the inner surface 233 of the second housing member 230 collectively define the inner volume 213 of the housing 218. As shown in FIG. 8, the arrangement of the inner surfaces 223 and 233 is such that the inner volume 213 has and/or defines a tortuous cross-sectional shape. For example, the inner volume 213 can have a substantially S-shaped or an at least partially S-shaped cross-sectional shape. More specifically, the inner surface 223 of the first housing member 220 includes and/or forms a ridge, tab, flange, protrusion, and/or wall configured to separate the first portion 224 of the inner surface 223 from the second portion 225 of the inner surface 223. Thus, the tortuous cross-sectional shape of the inner volume 213 forms and/or defines a first portion 214 of the inner volume 213 and a second portion 215 of the inner volume 213. In this manner, the first portion 214 of the inner volume 213 is spaced apart from the second portion 215 of the inner volume 213 without being fluidically isolated therefrom. In other words, the first portion 214 of the inner volume 213 defines an axis that is parallel to and offset from an axis defined by the second portion 215 of the inner volume 213.

As shown in FIG. 8, the first portion 214 of the inner volume 213 extends through a wall of the housing 218. Similarly stated, the housing 218 defines (e.g., the first housing member 220 and the second housing member 230 collectively define) a slot, channel, track, opening, and/or the like that is in fluid communication with the first portion 214 of the inner volume 213. Conversely, the second portion 215 of the inner volume 213 is entirely defined and/or enclosed (at least in the circumferential direction) by the housing 218. The tortuous cross-sectional shape of the inner volume 213 is such that the second portion 215 cannot be viewed (e.g., is out of the line of sight) via the slot (in fluid communication with the first portion 214 of the inner volume 213), which in turn, can limit and/or substantially prevent contamination of the catheter 260 disposed therein.

In this embodiment, the second portion 215 of the inner volume 213 is substantially aligned with, for example, a portion of the opening 217 and a portion of an opening defined by the coupler 216. Moreover, the second portion 215 of the inner volume 213 is configured to be substantially aligned with the lock 240 when the lock is coupled to the coupler 216 of the housing 218. In other words, the axis defined by the second portion 215 of the inner volume 213 is substantially co-axial with an axis defined by a portion of the lock 240, as described in further detail herein. In this manner, the second portion 215 of the inner volume 213 can movably receive, for example, a portion of the advancement member 270 and a portion of the catheter 260. Thus, the advancement member 270 can be moved relative to the housing 218 to move the catheter 260 between a first position, in which the catheter 260 is entirely disposed within the second portion 215 of the inner volume 213, and a second position, in which at least a portion of the catheter 260 extends outside of the second portion 215 of the inner volume 213 and distal to the housing 218, as described in further detail herein.

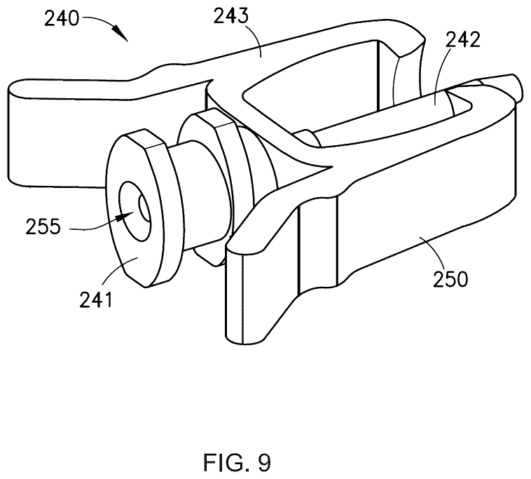

The lock 240 of the blood draw device 200 can be any suitable shape, size, and/or configuration. As described above, the lock 240 is configured to be physically and fluidically coupled to the housing 218 and configured to couple the introducer 210 to the PIVC and/or any suitable intermediate device or adapter coupled to the PIVC. The lock 240 has a coupler 241, a blunt cannula 242, a first arm 243, and a second arm 250, as shown in FIG. 9. In addition, the lock 240 defines a lumen 255 extending through the coupler 241 and the blunt cannula 242. The coupler 241 is configured to couple the lock 240 to the coupler 216 of the housing 218. Specifically, in this embodiment, the coupler 241 includes and/or forms one or more protrusions configured to selectively engage the threads defined and/or formed by the coupler 216 of the housing 218, thereby forming a threaded coupling.

The blunt cannula 242 extends from the coupler 246 and is disposed between the first arm 243 and the second arm 250. The blunt cannula 242 can be any suitable shape, size, and/or configuration. In some embodiments, the blunt cannula 242 can have a length that is sufficient to extend through at least a portion of the PIVC or through an adapter and at least partially into or through the PIVC. Furthermore, the blunt cannula 242 can have an inner diameter (a diameter of a surface at least partially defining the lumen 255) that is similar to or slightly larger than an outer diameter of a portion of the catheter 260. Thus, the lumen 255 of the lock 240 can receive a portion of the catheter 260 when the blood draw device 200 is transitioned between the first configuration and the second configuration.

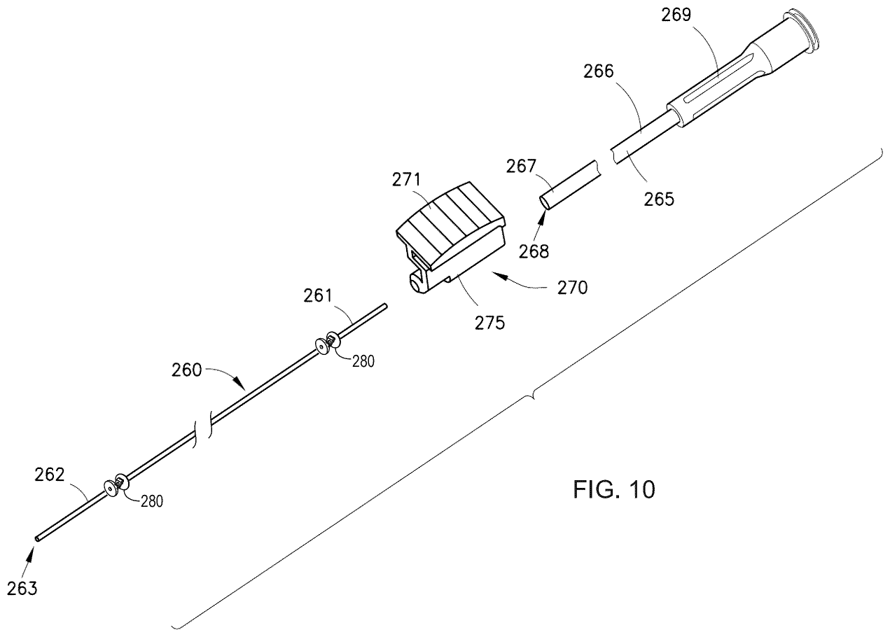

As described above, at least a portion of the catheter 260 and at least a portion of the secondary catheter 265 is movably disposed within the second portion 215 of the inner volume 213 defined by the housing 218. As shown in FIG. 10, the catheter 260 has a proximal end portion 261 and a distal end portion 262 and defines a lumen 263. The proximal end portion 261 of the catheter 260 is coupled to a second portion 275 of the advancement member 270. In this manner, the advancement member 270 can be moved relative to the housing 218 to move the catheter 260 between a first position, in which the catheter 260 is disposed within the housing 218 (e.g., the entire catheter 260 is disposed within the housing 218 or within the housing 218 and the lock 240) and a second position, in which the distal end portion of the catheter 260 is at least partially disposed in a position distal to the lock 240 and/or the PIVC (not shown) when the lock 240 is coupled to the PIVC, as described in further detail herein. The distal end portion 262 can be any suitable shape, size, and/or configuration and can define at least one opening in fluid communication the lumen 263.

The secondary catheter 265 has a proximal end portion 266 and a distal end portion 267 and defines a lumen 268. A portion of the secondary catheter 265 is disposed within and extends through the opening 217 of the housing 218 (e.g., collectively defined by the notches 223 and 233 of the first housing member 220 and second housing member 230, respectively). As such, the proximal end portion 266 is at least partially disposed outside of the housing 218 and the distal end portion 267 is at least partially disposed within the second portion 215 of the inner volume 213 defined by the housing 218. As described above, the secondary catheter 265 can be moved within the opening 217 between a first position and a second position to selectively clamp, pinch, kink, bend, and/or otherwise deform a portion of the secondary catheter 265, which in turn, obstructs, pinches, kinks, closes, seals, etc. the lumen 268 of the secondary catheter 265. For example, the first position can be associated and/or aligned with a first portion of the opening 217 having a larger perimeter and/or diameter than a perimeter and/or diameter of a second portion of the opening 217 associated and/or aligned with the second position. Thus, a user can manipulate the secondary catheter 265 to occlude the lumen 268 of the secondary catheter 265, thereby limiting, restricting, and/or substantially preventing a flow of a fluid therethrough.

As shown in FIG. 10, the proximal end portion 266 of the secondary catheter 265 is coupled to and/or otherwise includes a coupler 269. The coupler 269 is configured to physically and fluidically couple the secondary catheter 265 to any suitable device such as, for example, a fluid reservoir, fluid source, syringe, evacuated container holder (e.g., having a sheathed needle or configured to be coupled to a sheathed needle), pump, and/or the like. The distal end portion 267 of the secondary catheter 265 is at least partially disposed within the second portion 215 of the inner volume 213 defined by the housing 218 and is coupled to a second portion 275 of the advancement member 270. In some embodiments, the secondary catheter 265 can have a larger diameter than the catheter 260 such that the proximal end portion 261 of the catheter 260 is at least partially disposed within the lumen 268 defined by the secondary catheter 265 when the catheter 260 and the secondary catheter 265 are coupled to the second portion 275 of the advancement member 270. In some embodiments, such an arrangement can, for example, reduce and/or substantially prevent leaks associated with fluid flowing between the catheter 260 and the secondary catheter 265. In some embodiments, such an arrangement can also limit, reduce, and/or substantially prevent hemolysis of a volume of blood as the volume of blood flows through the catheter 260 and the secondary catheter 265. In this manner, when the coupler 269 is coupled to a fluid reservoir, fluid source, syringe, evacuated container, pump, etc., the secondary catheter 265 establishes fluid communication between the reservoir, source, pump, etc. and the catheter 260.

The advancement member 270 of the blood draw device 200 is coupled to the catheter 260 and can be moved along a length of the housing 218 to transition the blood draw device 200 between its first configuration, in which the catheter 260 is in the first position, and its second configuration, in which the catheter 260 is in the second position. The advancement member 270 can be any suitable shape, size, and/or configuration. For example, in some embodiments, the advancement member 270 can have a size and shape that is associated with and/or based at least in part on a size and/or shape of the housing 218.

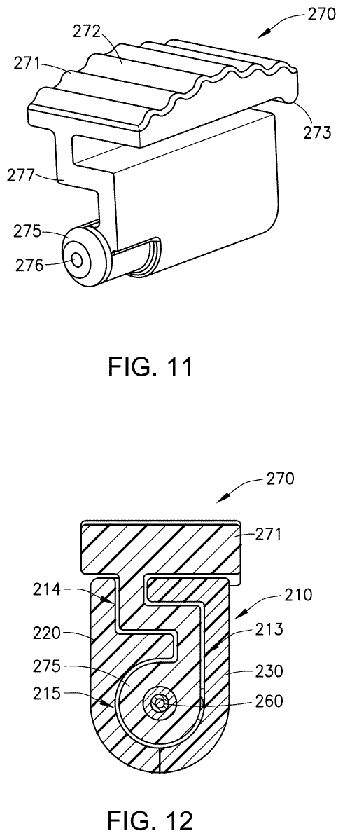

As shown in FIGS. 11 and 12, the advancement member 270 includes a first portion 271, the second portion 275, and a wall 277 extending therebetween. The first portion 271 of the advancement member 270 is at least partially disposed within the first portion 214 of the inner volume 213 defined by the housing 218 and the second portion 275 of the advancement member 270 is disposed within the second portion 215 of the inner volume 213, as described above. The first portion 271 of the advancement member 270 includes an engagement member 272. The arrangement of the advancement member 270 is such that the engagement member 272 is disposed outside of the housing 218 while the rest of the first portion 271 is within the first portion 214 of the inner volume 213 defined by the housing 218. As such, the engagement member 272 can be engaged and/or manipulated by a user (e.g., by a finger or thumb of the user) to move the advancement member 270 relative to the housing 218. In some embodiments, the engagement member 272 can include a set of ridges and/or any suitable surface finish that can, for example, increase the ergonomics of the advancement member 270 and/or blood draw device 200.

The engagement member 272 includes a tab 273 disposed at or near a proximal end portion of the engagement member 272. The tab 273 can be any suitable tab, rail, ridge, bump, protrusion, knob, roller, slider, etc. that extends from a surface of the engagement member 272. The tab 273 is configured to selectively engage the top surface 235 of the second housing member 230 of the housing 218. More specifically, the tab 273 is in contact with the ribs 236 formed by the second housing member 230 and moves along each successive rib as the advancement member 270 is moved along a length of the housing 218, with interaction of the tab 273 with the ribs 236 causing vibration of the advancement member 270.

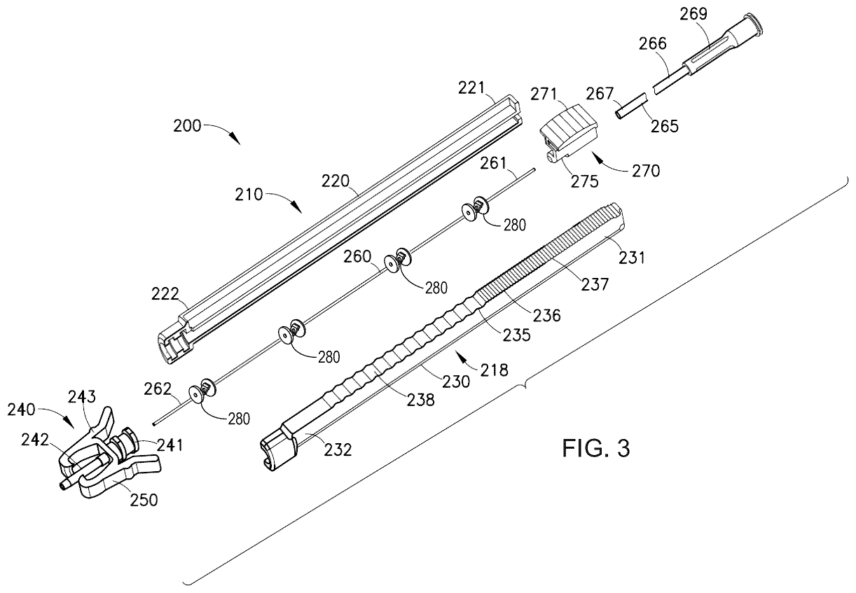

As shown in FIGS. 3 and 10, one or more catheter support inserts 280 (or, more generally, “support inserts”) of introducer 210 are positioned along a length of the catheter 260 between the proximal and distal ends thereof. As shown in FIG. 3, four support inserts 280 are positioned along a length of the catheter 260, but it is recognized a lesser or greater number of support inserts 280 could be provided in introducer—including only a single support insert 280 positioned on catheter 260, or two, three, five, or more support inserts 280 potentially being positioned along a length of the catheter 260. With multiple support inserts 280 being provided, the support inserts 280 may be spaced equidistantly along the catheter 260.

The support inserts 280 of introducer 210 are positioned within the housing 218 (i.e., within the second portion 215 of the inner volume 213) at locations between the advancement member 270 and the distal end 212 of the introducer 210. The catheter 260 is routed through a passageway of each of the support inserts 280 to reduce an unsupported length of the catheter 260, effectively increasing the resistance the catheter 260 can withstand before buckling or kinking. That is, the force at which a catheter 260 buckles/kinks is inversely proportional to the effective length squared. Thus, as the effective length of the catheter 260 increases, the force required to buckle/kink the catheter 260 decreases, making the catheter more prone to buckling/kinking. The support inserts 280 support the catheter 260, thereby shortening the effective length of the catheter 260, increasing the force required to buckle/kink the catheter 260, and decreasing the tendency of the catheter 260 to buckle/kink—i.e., the support inserts 280 divide the unsupported length of the catheter 260 into smaller portions that are more resistant to bending, kinking, flexing, and/or deformation.

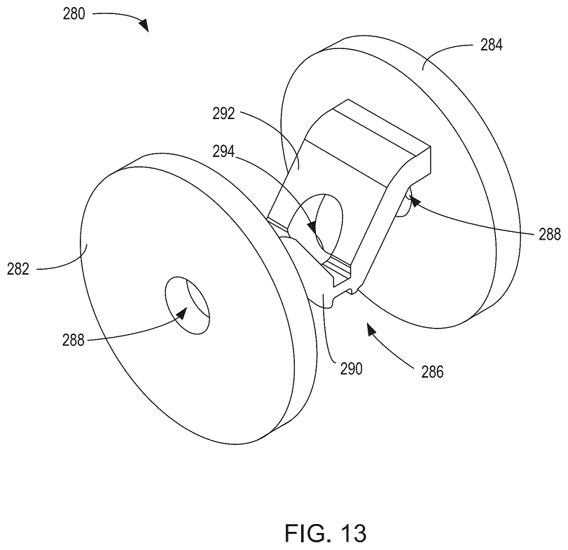

Referring now to FIGS. 13-15, a support insert 280 is shown in further detail, in accordance with an embodiment of the disclosure. The support insert 280 may be formed as a single, integrally formed component that generally includes a first end disc 282, a second end disc 284, and a hinge member 286 disposed between the first end disc 282 and the second end disc 284 that connects the first end disc 282 to the second end disc 284. In some embodiments, the support insert 280 may be formed of a moldable material that provides for easy manufacturing thereof, with the support insert 280 being formed of a polymeric material that may be injection molded, as a non-limiting example.

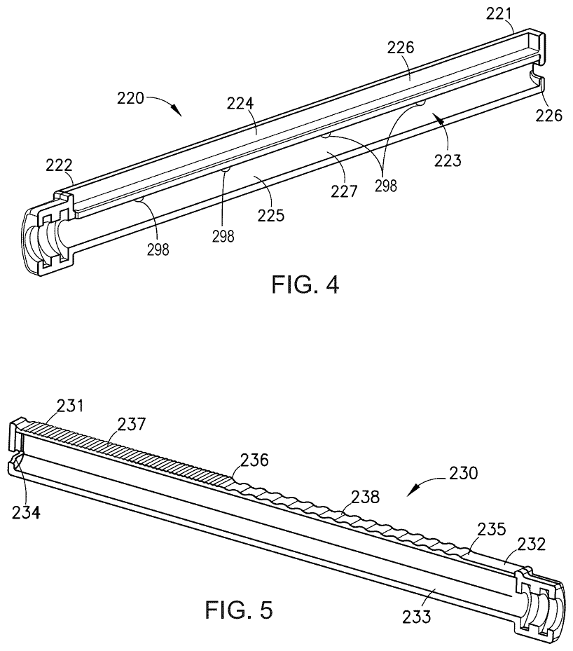

Each of the first end disc 282 and the second end disc 284 may have a cross-sectional shape that is based at least in part on a cross-sectional shape of the second portion 215 of the inner volume 213 defined by the introducer 210, for example, at least a partially circular cross-sectional shape. In this manner, the inner surface 223 of the first member 220 (FIG. 4) and the inner surface 233 of the second member 230 (FIG. 5) can support and/or guide the support insert 280 as the support insert 280 is moved relative to the introducer 210, as explained in further detail below. Each of the first end disc 282 and the second end disc 284 includes an opening 288 formed therethrough, such as at a center point thereof. The openings 288 are sized and shaped to allow the catheter 260 to freely pass therethrough, while being contained thereby to prevent excessive movement of the catheter 260 within the openings 288.

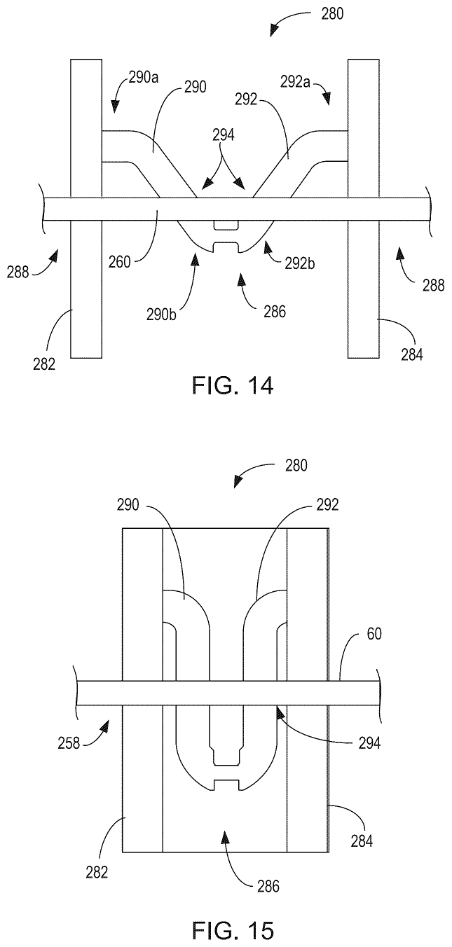

According to aspects of the disclosure, the hinge member 286 connecting the first end disc 282 to the second end disc 284 is configured as a living hinge (hereafter “living hinge 286”) that is formed integrally with the first end disc 282 and the second end disc 284. The living hinge 286 is formed of a first leg 290 and a second leg 292 that are joined together, with the first leg 290 having a first end 290a and a second end 290b and the second leg 292 having a first end 292a and a second end 292b. The first end 290a of the first leg 290 is joined to the first end disc 282, while the first end 292a of the second leg 292 is joined to the second end disc 284, and the second end 290b of the first leg 290 and the second end 292b of the second leg 292 are joined together. The first leg 290 and the second leg 292 of the living hinge 286 may form a V-shape when in an at-rest position, and the living hinge 286 is configured to flex responsive to a force being applied to the catheter support insert. That is, when a compressive force (e.g., a pushing force applied by advancement member 270 as it is moved distally along housing 218) is applied to the catheter support insert 280, the living hinge 286 is actuated and may transition/flex from its initial V-shape (FIG. 14) to a shape where the first leg 290 and the second leg 292 are parallel (or substantially parallel) to each other, up until the first leg 290 and the second leg 292 may be brought flush against one another (FIG. 15). With the living hinge 286 flexing as such, the support insert 280 transitions from an “extended state” to a “compressed state,” thereby reducing a distance between the first end disc 282 and the second end disc 284 and shortening the overall length of the catheter support insert 280. In some embodiments, the length of the catheter support insert 280 may be reduced from about 5.0 mm when in its extended state down to about 2.0 mm when in its compressed state.

According to some embodiments, each of the first leg 290 and the second leg 292 includes an opening 294 formed therein through which the catheter 260 is routed. Similar to the openings 288 in the first and second end discs 282, 284, the openings 294 in the first and second legs 290, 292 are sized and shaped to allow the catheter 260 to freely pass therethrough, while being contained thereby to prevent excessive movement of the catheter 260 within the openings 294. The opening 294 are formed at locations in the legs 290, 292 such that they are aligned with each other and with the openings 288 in the first and second end discs 282, 284.

The arrangement of the openings 288 formed in the first and second end discs 282, 284 and the openings 294 in the legs 290, 292 of the living hinge 286 collectively define a passageway 296 of the catheter support insert 280 through which the catheter 260 passes. The catheter 260 may be supported by the catheter support insert 280 along a length thereof that extends through the passageway 296, to reduce an unsupported length of the catheter 260 and effectively increase the resistance the catheter 260 can withstand before buckling. That is, the force at which a catheter 260 buckles is inversely proportional to the effective length squared. Thus, as the effective length of the catheter 260 increases, the buckling force required to buckle the catheter 260 decreases, making the catheter more prone to buckling. The catheter support insert 280 supports the catheter 260, thereby shortening the effective length of the catheter 260, increasing the force required to buckle the catheter 260, and decreasing the tendency of the catheter 260 to buckle—i.e., the catheter support insert 280 divides the unsupported length of the catheter 260 into two smaller portions that are more resistant to bending, kinking, flexing, and/or deformation.

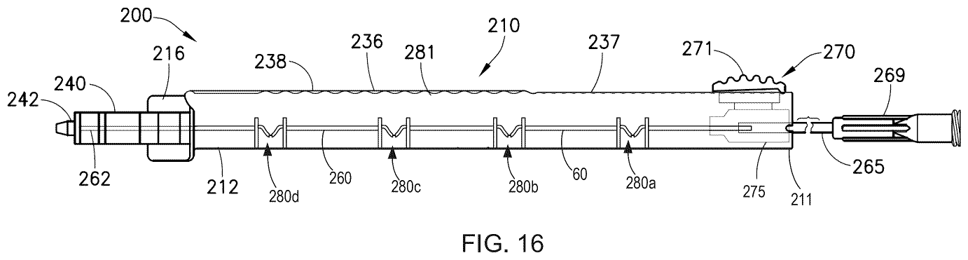

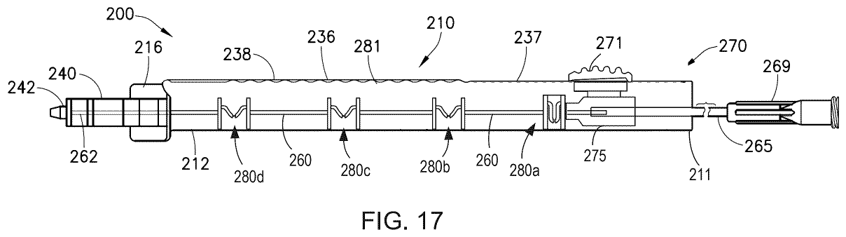

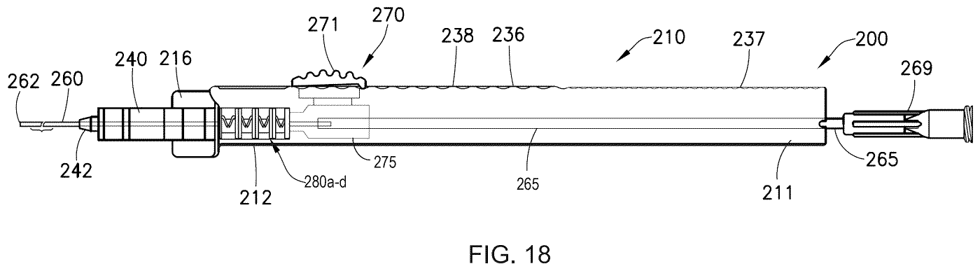

Referring now to FIGS. 16-18, changing of the blood draw device 200 from the first configuration to the second configuration is shown, with an intermediate configuration between the first configuration and the second configuration also shown. The blood draw device 200 can be in the first configuration prior to use and can be transitioned by a user (e.g., a doctor, physician, nurse, technician, phlebotomist, and/or the like) from the first configuration (FIG. 16), to the intermediate configuration (FIG. 17), and finally to the second configuration (FIG. 18) to dispose at least the distal end portion 262 of the catheter 260 in a distal position relative to the housing 218 (e.g., within an indwelling PIVC (not shown) or distal to the indwelling PIVC).

The blood draw device 200 is in the first configuration of FIG. 16 when the catheter 260 is disposed in the first position within the housing 218. In some embodiments, substantially the entire catheter 260 is disposed within the housing 218 when the catheter 260 is in the first position. In such embodiments, the housing 218 can include a seal or the like (as described above) that can substantially seal the distal end portion 212 of the housing 210 to isolate the catheter 260 within the second portion 215 of the inner volume 213. In other embodiments, the catheter 260 is disposed within the housing 210 and the lock 240 when catheter 260 is in the first position.

With the blood draw device 200 in the first configuration, the advancement member 270 is disposed toward the proximal end portion 211 of the introducer 210. The proximal end portion 261 of the catheter 260 is coupled to the second portion 275 of the advancement member 270, and the catheter 260 extends through the second portion 215 of the inner volume 213 of the introducer 210 and through the passageway 285 of each of the catheter support inserts 280 (such as the four support inserts 280 shown in FIGS. 16-18), with the distal end portion 262 of the catheter 260 being received in the lumen of the coupler 216 and/or the lumen 255 of the lock 240.

With the blood draw device 200 in the first configuration, the catheter support inserts 280 are positioned in their initial pre-use position, such as being spaced apart equidistantly along the unsupported portion of the catheter 260 (extending from the second portion 275 of the advancement member 270 to the distal end portion 212 of the introducer 210). In some embodiments, the catheter support inserts 280 are retained in this pre-use position by bump projections 298 that are formed on the inner surface of the introducer housing (i.e., inner surface 223 of first housing member 220). The bump projections 298 protrude inwardly from the inner surface 223 of the housing 218 and may be formed at locations thereon corresponding to the pre-use positions of the catheter support inserts 280. The bump projections 298 may engage one of the end discs 282, 284 of each catheter support insert 280, with the catheter support inserts 280 ideally positioned such that the respective bump projections 298 are positioned between the first and second end discs 282, 284 of the catheter support inserts 280—such that both proximal and distal movement of the catheter support inserts 280 is restrained by the bump projections 298.

To transition from the first configuration toward the second configuration, the user engages the engagement member 272 of the first portion 271 of the advancement member 270 to move the advancement member 270 relative to the housing 218, which in turn, moves the catheter 260 from the first position (e.g., disposed within the housing 218) toward the second position. In this manner, the catheter 260 is moved through the second portion 215 of the inner volume 213, the passageways 285 of the catheter support inserts 280, and the lumen 255 of the lock 240 and, as such, at least the distal end portion 262 of the catheter 260 is disposed outside of and distal to the lock 240.

After partial movement of the advancement member 270 in the distal direction, the blood draw device 200 is considered to be in an intermediate configuration as shown in FIG. 17. With the blood draw device 200 in an intermediate configuration, the advancement member 270 contacts a proximal-most catheter support insert 280a. As the advancement member 270 is advanced further in the distal direction, the advancement member 270 pushes the proximal-most catheter support insert 280a in the distal direction. According to aspects of the disclosure, the pushing of the catheter support insert 280a by the advancement member 270 is sufficient to force the catheter support insert 280a past a bump projection 298 on the housing 218 that held the catheter support insert 280a in its pre-use position.

As the proximal-most catheter support insert 280a is being pushed by the advancement member 270, the advancement member 270 also applies a pressure against the catheter support insert 280a that causes the catheter support insert 280a to transition (at least partially) from its extended state to its compressed state. That is, the pressure applied by the advancement member 270 against the catheter support insert 280a causes the living hinge 286 to collapse, therefore bringing the first end disc 282 and second end disc 284 closer together.

With the blood draw device 200 remaining in the intermediate configuration, continued advancement of the advancement member 270 in the distal direction causes the proximal-most catheter support insert 280a to eventually come into contact with the next catheter support insert 280b positioned on the catheter 260. With the proximal-most catheter support insert 280a brought into contact with the next catheter support insert 280b and the advancement member 270 continuing to advance, the advancement member 270 and catheter support insert 280a push the next catheter support insert 280b in the distal direction (past its engagement with a bump projection 298). Again, as advancement of the next catheter support insert 280b is occurring, the next catheter support insert 280b is transitioned (at least partially) from its extended state to its compressed state.

As the advancement member 270 continues to advance and the blood draw device 200 continues to transition from the intermediate configuration toward the second configuration, the process described for advancement and compression of the catheter support inserts 280a, 280b is repeated for all additional catheter support inserts included in the introducer 210—i.e., third and fourth catheter support inserts 280c, 280d in the illustrated embodiment. Advancement of the advancement member 270 and catheter support inserts 280a-d continues until the advancement member 270 and catheter support inserts 280a-d are moved to a distal most position within housing 218, with the blood draw device 200 then considered to be in the second configuration as shown in FIG. 18. In this position, the advancement member 270 will have pushed the catheter support inserts 280a-d to the distal end portion 212 of the housing 218, with the plurality of catheter support inserts 280a-d each in their compressed state and in contact with each other in a stacked arrangement. In some embodiments, a length of the stacked arrangement of the four catheter support inserts 280a-d is between 11.0 and 12.0 mm, as a non-limiting example.

With the blood draw device 200 in the second configuration, the advancement member 270 and catheter support inserts 280a-d are moved to a distal most position within housing 218 and the catheter 260 is thus moved to its second position, where the distal end portion 262 thereof is placed in a desired position relative to a distal end portion of the PIVC. In some instances, for example, a distal end 262 of the catheter 260 can be substantially flush with a distal end of the PIVC when the catheter 260 is in the second position. In other instances, the distal end 262 of the catheter 260 can extend a predetermined distance beyond the distal end of the PIVC (e.g., distal to the distal end of the PIVC), such that the distal end 262 of the catheter 260 is positioned within the vein at a predetermined distance beyond the distal surface of the catheter 260 (e.g., a position within a vein that is substantially free from debris (e.g., fibrin/blood clots) otherwise surrounding the distal end portion of the PIVC).

With the catheter 260 in the second position, the user can establish fluid communication between a fluid reservoir, fluid source, syringe, and/or the like and the catheter 260, With the catheter 260 in fluid communication with the fluid reservoir and/or fluid source, the blood draw device 200 can then aspirate a volume of blood from the vein based at least in part on disposing the distal surface of the catheter 260 at the predetermined and/or desired distance beyond the distal surface of the PIVC. In some instances, once a desired amount of blood has been collected and/or once a desired volume of a drug has been delivered to the patient, the user can move the advancement member 270 in the proximal direction, such as moving the advancement member 270 back to its proximal most position, so as to move the catheter 260 back to the first position.

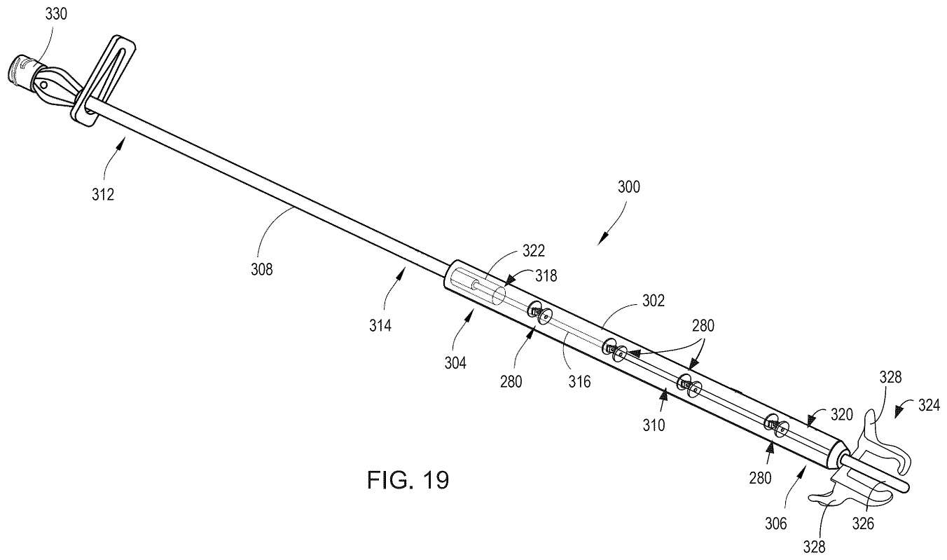

It is recognized that aspects of the disclosure are not limited to the specific blood draw device 200 shown and described in FIGS. 1-18, and that blood draw devices with other suitable constructions may also incorporate aspects of the disclosure. Referring now to FIG. 19, a blood draw device 300 is shown according to another embodiment of the disclosure. The blood draw device 300 includes a housing 302 having a proximal end 304 and a distal end 306, and an advancement member 308 slideably received within housing 302 (i.e., within an inner volume 310 of housing 302). In the illustrated embodiment, the advancement member 308 is provided as a telescopic cylinder that is provided in a telescoping relationship with housing 302, such that advancement member 308 may be slideably received entirely, or almost entirely, within the inner volume 310 of housing 302. Advancement member 308 also includes a proximal end 312 and a distal end 314 and, in non-limiting embodiments, advancement member 308 may have a variable diameter along its length. As one example, the distal end 314 of advancement member 308 may have a larger diameter than other portions of advancement member 308 such that, as advancement member 308 is retracted, one or more features on housing 302 may interact with the enlarged portion of advancement member 308 to prevent pulling advancement member 308 completely out of housing 302.

Blood draw device 300 further includes a catheter 316 having a proximal end 318 and a distal end 320. Catheter 316 is received within the inner volume 310 of housing 302, and may be advanced and/or retracted relative to housing 302 by displacement of the advancement member 308 relative to the housing 302. In some embodiments, the catheter 316 may be joined to advancement member 308 via a fitting 322 provided at the distal end 314 of advancement member 308, such that displacement of the advancement member 308 relative to the housing 302 causes a corresponding displacement of catheter 316. In non-limiting embodiments, catheter 316 may be advanced from a first positon in which distal end 320 of catheter 316 is within housing 302, to a second position in which a distal end 320 of catheter 316 is positioned distally of housing 302 (and also positioned distally of catheter 316), as previously described regarding blood draw device 200 and operation thereof.

Blood draw device also includes a coupling device 324 thereon which may be identical to the coupling device 240 shown and described in the blood draw device of FIGS. 1-18. That is, coupling device 324 is configured as a lock 324 that includes a blunted cannula 326 and locking arms 328 for coupling to an access connector 46 of a PIVC, with the blunted cannula 326 and locking arms 328 forming three points of contact therewith. However, it is appreciated that alternative embodiments of blood draw device 300 may include a coupling device 324 of another type to secure blood draw device 300 to an access connector, including luer connections, clips, blunt plastic cannulae, blunt metal cannulae, hybrid luers (e.g., with a cannula) friction fits, and the like. According to aspects of the disclosure, a secondary catheter (not shown) may be routed through the telescopic cylinder of advancement member 308, with the secondary catheter providing a fluid connection between the catheter 316 and a coupler 330 provided at the proximal end portion of the secondary catheter—with the coupler 330 configured to mate with a collection device (not shown).

As described in detail above, blood draw device 300 may incorporate one or more catheter support inserts 280 therein that are configured to provide support along a length of the catheter 316, so as to effectively increase the resistance the catheter 316 can withstand before buckling or kinking. As previously described, each of the catheter support inserts 280 includes a first end disc 282, a second end disc 284, and a living hinge 286—with openings 288, 294 formed in the end discs 282, 284 and arms 290, 292 of the living hinge 286 to define a passageway 296 through which the catheter 316 is routed, so as to be supported by the support insert 280. The support insert 280 may be pushed within the housing 302 as the advancement member 308 is moved distally through the housing 302, with the support inserts 280 compressing and being moved into a stacked arranged at the distal end 306 of housing 302 upon the advancement member 308 being advanced fully into the housing 302.

Beneficially, embodiments of the disclosure are directed to a blood draw device that provides for advancement of a catheter into a PIVC while minimizing the potential for bending, kinking, or deformation of the catheter. The blood draw device includes an introducer housing having one or more catheter support inserts positioned therein and along a length of the catheter, with the catheter routed through a passageway of each of the support inserts to reduce an unsupported length of the catheter. With the catheter support insert(s) supporting the catheter at one or more locations within the introducer housing, the unsupported length of the catheter is divided into smaller portions, such that the effective length of the catheter is shortened and the resistance the catheter can withstand before buckling or kinking is effectively increased.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

What is claimed is:

1. A blood draw device for advancing a catheter into an indwelling catheter of a vascular access device, the blood draw device comprising:
an introducer comprising a housing having a proximal end portion and a distal end portion, with the distal end portion coupleable to the vascular access device, and with the housing defining an inner volume configured to movably receive the catheter;
an advancement member movably coupled to the housing and configured to move relative to the housing to move the catheter between a first position, in which the catheter is disposed within the introducer, and a second position, in which a distal end portion of the catheter is disposed beyond the distal end portion of the introducer, such that at least a first portion of the catheter may be disposed within the indwelling catheter; and
one or more catheter support inserts positioned within the inner volume and positioned between the advancement member and the distal end portion of the housing, each of the one or more catheter support inserts defining a passageway therein that receives the catheter to provide support to a length of the catheter that extends between the advancement member and the distal end portion of the housing;
wherein each of the one or more catheter support inserts comprises:
a first end disc and a second end disc each including an opening therein through which the catheter is routed; and
a hinge member disposed between the first end disc and the second end disc and connecting the first end disc to the second end disc, the hinge member being flexible so as to vary a distance between the first end disc and the second end disc.

2. The blood draw device of claim 1, wherein the hinge member comprises a living hinge formed integrally with the first end disc and the second end disc.

3. The blood draw device of claim 2, wherein the living hinge comprises:
a first leg including a first end and a second end; and
a second leg including a first end and a second end;
wherein the first end of the first leg is joined to the first end disc and the first end of the second leg is joined to the second end disc; and
wherein the second end of the first leg and the second end of the second leg are joined together, with the first leg and the second leg forming a V-shape when in an at-rest position.

4. The blood draw device of claim 3, wherein each of the first leg and the second leg includes an opening formed therein through which the catheter is routed, and wherein the openings in the first and second legs and the openings in the first and second end discs collectively define the passageway of the catheter support insert.

5. The blood draw device of claim 1, wherein for a respective catheter support insert of the one or more catheter support inserts that is nearest the advancement member, the advancement member engages the catheter support insert during movement thereof when moving the catheter from the first position to the second position, with the advancement member engaging and moving the catheter support insert with respect to the housing.

6. The blood draw device of claim 5, wherein as the advancement member moves the catheter support insert with respect to the housing, the hinge member flexes to transition the catheter support insert from an extended state to a compressed state, thereby shortening a length of the catheter support insert.

7. The blood draw device of claim 6, wherein in the extended state, the catheter support insert has a length of 5 mm.

8. The blood draw device of claim 5, wherein the advancement member comprises:
a first portion comprising an engagement portion movable along a top surface of the housing; and
a second portion positioned at least partially within the inner volume and joined to the first portion through a slot formed in the top surface of the housing;
wherein the second portion engages the catheter support insert as the advancement member is moved distally along the housing.

9. The blood draw device of claim 5, wherein the advancement member comprises a telescopic cylinder configured to slide distally into the inner volume of the housing, the telescopic cylinder including a fitting at a distal end thereof that engages the catheter support insert as the telescopic cylinder is moved distally into the housing.

10. The blood draw device of claim 1, wherein the one or more catheter support inserts comprises a plurality of catheter support inserts spaced apart along a length of the catheter between the advancement member and the distal end portion of the housing.

11. The blood draw device of claim 10, wherein the plurality of catheter support inserts are spaced apart equidistantly along the length of the catheter.

12. The blood draw device of claim 1, wherein with the catheter in the second position, the advancement member moves the plurality of catheter support inserts to the distal end portion of the housing, with the plurality of catheter support inserts in contact with each other in a stacked arrangement.

13. The blood draw device of claim 12, wherein a length of the stacked arrangement of the plurality of catheter support inserts is between 11.0 and 12.0 mm.

14. The blood draw device of claim 1, wherein the housing comprises an inner surface including one or more bump projections formed thereon, with a respective bump projection of the one or more bump projections configured to engage a respective catheter support insert of the one or more catheter support inserts to retain the catheter support insert in-place within the housing.

15. The blood draw device of claim 14, wherein the catheter support insert is pushed past the bump projection upon the advancement member engaging and moving the catheter support insert, as the advancement member moves the catheter from the first position to the second position.

16. A blood draw device for advancing a catheter into an indwelling catheter of a vascular access device, the blood draw device comprising:
an introducer comprising a housing having a proximal end portion and a distal end portion, with the distal end portion coupleable to the vascular access device, and with the housing defining an inner volume configured to movably receive the catheter;
an advancement member movably coupled to the housing and configured to move relative to the housing to move the catheter between a first position, in which the catheter is disposed within the introducer, and a second position, in which a distal end portion of the catheter is disposed beyond the distal end portion of the introducer, such that at least a first portion of the catheter may be disposed within the indwelling catheter; and
a plurality of catheter support inserts positioned within the inner volume between the advancement member and the distal end portion of the housing, the plurality of catheter support inserts spaced apart along a length of the catheter, with each of the plurality of catheter support inserts defining a passageway therethrough that receives the catheter;
wherein, with the advancement member moved distally relative to the housing to move the catheter from the first position to the second position, the advancement member pushes the plurality of catheter support inserts to the distal end portion, with the plurality of catheter support inserts positioned in a stacked arrangement.

17. The blood draw device of claim 16, wherein each of the plurality of catheter support inserts comprises a compressible member that transitions from an extended state to a compressed state when a distally directed force is applied thereto.

18. The blood draw device of claim 17, wherein with the plurality of catheter support inserts positioned in the stacked arrangement, all of the plurality of catheter support inserts are in contact with each other and are in the compressed state.

19. The blood draw device of claim 18, wherein a length of the stacked arrangement of the plurality of catheter support inserts is between 11.0 and 12.0 mm.

20. The blood draw device of claim 16, wherein each of the plurality of catheter support inserts comprises:
a first end disc and a second end disc each including an opening therein through which the catheter is routed; and
a living hinge disposed between the first end disc and the second end disc and connecting the first end disc to the second end disc, the living hinge being actuatable to transition the catheter support insert from the extended state to the compressed state;
wherein the living hinge comprises a first leg and a second leg connected together, with each of the first leg and the second leg including an opening formed therein through which the catheter is routed, and wherein the openings in the first and second legs and the openings in the first and second end discs collectively define the passageway of the catheter support insert.
